(19) United States
(12) Patent Application Publication
(10) Pub. No.: US 2007/0134189 A1
(43) Pub. Date: Jun. 14, 2007

(54) Anti-wrinkle cosmetic

(75) Inventors: Karin Golz-Berner, Monacco (MC); Leonhard Zastrow, Monaco (MC); Olivier Doucet, Villefranche s/Mer (FR)

Correspondence Address: STEPHAN A. PENDORF, P.A., PENDORF & CUTLIFF, 5111 MEMORIAL HIGHWAY, TAMPA, FL, 33634, US

(73) Assignee: Coty B.V.

(21) Appl. No.: 10/588,748

(22) PCT Filed: Feb. 10, 2005
(86) PCT No.: PCT/EP05/01460

§ 371 Date: Aug. 9, 2006

(30) Foreign Application Priority Data
Feb. 11, 2004 (DE) 10 2004 007 385.6

(51) Int. Cl. A61K 8/97; A61K 8/64

(52) U.S. Cl. 424/70.14; 424/74

(57) ABSTRACT

The invention relates to a novel anti-wrinkle cosmetic based on natural plant extracts. Said cosmetic comprises, in a W/O silicon system, between 0.05 and 2 wt. % of a Passiflora extract, and respectively between 0.05 and 3 wt. % of Papaver, Metha and Myrtus extracts, along with standard cosmetic auxiliary agents, carriers and active ingredients. In addition to a silky texture, said cosmetic has a long-lasting moistening effect, and an improved elasticity characteristic.

### CROSS-REFERENCE TO RELATED APPLICATION

This application is a national stage of PCT/EP2005/001460 filed Feb. 10, 2005 and based upon DE 10 2004 007 385.6 filed Feb. 11, 2004 under the International Convention.

### BACKGROUND OF THE INVENTION

1. Field of the Invention

The invention relates to a novel anti-wrinkle cosmetic based on natural plant extracts.

It is known to use plant extracts in cosmetic products. Surprisingly, the overall effect resulting from combinations of several plant extracts is often different from the individual effects and also depends on the parts of the plants selected and the extractants used. Poppy (Papaver) is in most cases known for its internal effects only, due to its alkaloid content. Passiflora extracts have, in general, also been used internally as an anti-spasm or anti-inflammatory agent. EP 1002524 B1 describes a mixture of musk rose oil, camellia oil and sunflower oil, which can also contain passion flower oil, as an anti-ageing agent. Mint (Mentha) has in particular been used externally so far, for treating irritations of the skin or, combined with other plants, for skin-whitening (WO 02/065999) WO 03/083028 describes a body cleanser containing Mentha spicata var. viridis as active agent.

2. Description of Related Art

The object of the invention is to provide a cosmetic for reducing wrinkles which at the same time has a silky texture when being applied, without the additives normally used for this purpose, improves the skin's elasticity and has a long-lasting moisturizing effect.

### SUMMARY OF THE INVENTION

According to the invention, the anti-wrinkle cosmetic consists of a w/O silicone oil system and comprises the following active ingredients (in % by weight and relative to the cosmetic's total weight)
0.05 to 3% of an extract from Papaver,
0.05 to 2% of an extract from Passiflora,
0.05 to 3% of an extract from Mentha,
0.05 to 3% of an extract from Myrtus,
   in addition to usual cosmetic auxiliaries, carriers, active agents or mixtures thereof, which make up the remainder up to 100%.

It has been found that a combination of the four aforesaid specific plant extracts has an anti-wrinkle effect on human skin and also enables significant improvements as regards fine lines in elderly skin.

In addition to the active ingredients mentioned above, the cosmetic can preferably contain 0.5 to 15% by weight of a solution of the hexapeptide acetyl-Glu-Glu-Met-Gln-Arg-Arg-NH2, the concentration of the pure peptide in said solution being 0.5 g/l. Contents of 0.5-2.0% by weight are particularly preferred. Surprisingly, the anti-wrinkle effect of the inventive combination of plant extracts is enhanced by the addition of the aforesaid hexapeptide, which probably inhibits the excessive production of catecholamines to which the formation of wrinkles can be put down.

It is preferred that the conditioned extract from Papaver be an extract from P. rhoeas, P. adulis, P. incamata, P. laurifolia, P. quadrangularis, P. somniferum or mixtures thereof. Said Papaver extract is an extract obtained from poppy seeds using a polyvalent alcohol, such as e.g. propylene glycol, at 20-50° C., optionally in a vacuum.

Further, it is preferred that the conditioned extract from Mentha be an extract from Mentha aquatica, Mentha arvensis, Mentha piperita, Mentha pulegium, Mentha rotundifolia, Mentha viridis or mixtures thereof containing Mentha in an amount of approx. 0.5% by weight relative to the total weight of the conditioned extract, which can additionally contain water, stabilizers and preservatives (INCI: Water & Mentha piperita leaf extract). The extraction is done at 20-50° C.

The extracts from Passiflora, e.g. P. incarnata, and from Myrtus, e.g. M. communis, M. communis ‘Flore Pleno’, M. communis ssp. tarentia, are extracts obtained from the flowers using propylene glycol (PPG) at temperatures of 20-50° C. The specific amounts contained in the conditioned extract are approx. 2-3% by weight relative to the total weight of the conditioned extract, which can additionally contain water, PPG and preservatives (INCI: Water & Passiflora incarnata flower extract and Water & Myrtus communis flower extract, respectively)

The myrtle extract (e.g. Myrtus communis) is an aqueous-alcoholic or purely alcoholic extract from flowers and leaves, the alcohol used being a polyvalent alcohol, such as propylene glycol, and the extraction being done at 20-50° C.

The texture of the inventive cosmetic is such that the 0/W emulsion transforms into a gel when being applied to and rubbed slightly into the skin, bringing about a particularly soft feel. There is no stickiness at all. Overall, a kind of Botox effect is achieved, which, however, manifests itself in a reduction and flattening of wrinkles and makes the skin look young.

In addition, it has been found that the skin's elasticity improved by up to 40% following 4 weeks of treatment. A test lasting for 8 hours and a long-term study carried on for 4 weeks showed that much moisture is retained, so that on the whole a significant long-lasting effect has been found for this composition of the invention.

Altogether, a skin-smoothing effect has been observed for the inventive cosmetic, which could not be expected from the individual ingredients. In particular, the silky texture is achieved without adding the usual pigments or powders, i.e. these can be omitted completely.

The inventive cosmetic further contains cosmetic auxiliaries and carriers as they are commonly used in such preparations, e.g. water, preservatives, colourants, thickeners, fragrances, alcohols, polyols, esters, electrolytes, gel-forming agents, polar and non-polar oils, polymers, copolymers, emulsifiers, waxes, stabilizers.

It is particularly preferred that antioxidants and free radical scavengers be added. These substances include vitamins such as vitamin C and derivatives thereof, e.g. ascorbyl acetate, ascorbyl phosphate and ascorbyl palmitate; vitamin A and derivatives thereof; folic acid and derivatives thereof; vitamin E and derivatives thereof such as tocopheryl acetate; flavons or flavonoids; amino acids such as histidine, glycine, tyrosine, tryptophan and derivatives thereof; carotenoids and carotenes such as e.g. σ-carotene, β-carotene; uric acid and derivatives thereof; σ-hydroxy acids such as citric acid, lactic acid, malic acid; stilbenes and derivatives thereof; and mixtures of the aforesaid substances.

In particular, the inventive silicone oil system consists of a combination of silicone oils with a silicone gel. Silicone oils which are advantageously used are e.g. cyclohexasiloxane, cyclopentasiloxane, cyclotetrasiloxane, dimethicone and mixtures thereof.

Silicone oils preferably make up between 5 and 15% by weight relative to the cosmetic's total weight.

Combinations of these silicone oils with silicone gels are e.g. those with Dimethicone & PEG 10 Dimethicone Crosspolymer & PEG 15 Dimethicone Crosspolymer (73:13.5:13.5) or with Dimethicone & PEG 10 Dimethicone Crosspolymer & PEG 15 Dimethicone Crosspolymer (73:13.5:13.5).

The inventive cosmetic can contain emulsifiers for preparing the 0/W emulsions. Suitable emulsifiers for 0/W emulsions are e.g. addition products of 2-30 moles ethylene oxide to linear C₈-C₂₂ fatty alcohols, to C₁₂-C₂₂ fatty acids and to C₈-C₁₅ alkyl phenols; C₁₂-C₂₂ fatty acid monoesters and diesters of addition products of 1-30 moles ethylene oxide to glycerine.

As described above, no pigments need to be added. If desired, however, pigments, pigment mixtures or powders having a pigment-like effect can be added as cosmetic auxiliaries, but this is not preferred. These also include those with a pearlescent effect. These pigments or powders can e.g. be silicon dioxide, iron oxides, natural aluminosilicates such as ochre, titanium (di)oxide, mica, kaolin, clays containing manganese such as umber and red bole, calcium carbonate, talcum, mica-titanium oxide, mica-titanium oxide-iron oxide, bismuth oxychloride, nylon globules, ceramic globules, expanded and non-expanded synthetic polymer powders, powdery natural organic compounds such as ground solid algae, ground parts of plants and mica-titanium oxide-organic colourant.

Polyols can also be contained in the inventive cosmetic. These are e.g. propylene glycol, dipropylene glycol, ethylene glycol, isoprene glycol, glycerine, butylene glycol, sorbitol and mixtures thereof. The polyol makes up between 0.1 and 40% by weight, preferably between approx. 5% and approx. 20% by weight of the gel composition.

Another additive for the inventive cosmetic is an active preparation with a high radical protection factor, containing a product obtained by extracting the bark of Quebracho blanco and subsequent enzymatic hydrolysis, which product contains at least 90% by weight proanthocyanidine oligomers and max. 10% by weight gallic acid, in microcapsules, and a silkworm extract obtained by extraction, which extract contains the peptide cecropine, amino acids and a vitamin mixture, and a non-ionic, cationic or anionic hydrogel or hydrogel mixture, and one or several phospholipid(s) and water. This is e.g. an active complex according to Example 1 or 2 of WO 99/66881 or e.g. an active complex according to Example 1 of WO 01/26617. Surprisingly, the addition of the aforesaid active preparation to the inventive combination considerably improves the effect of the latter as regards its free radical scavenging properties (e.g. radical protection factor RPF of the active complex of Example 2 of WO 99/66881=2,120; RPF of the inventive extract combination in Example 1=840; RPF of the active complex plus the extract combination =3,230 radicals.1O ¹⁴/mg).

Moreover, the inventive cosmetic can advantageously contain suitable water- and/or oil-soluble UVA or UVB filters or both. Advantageous oil-soluble UVB filters include derivatives of 4-aminobenzoic acid such as 4-(dimethylamino)-benzoic acid-(2-ethylhexyl) ester; esters of cinnamic acid such as 4-methoxy cinnamic acid (2-ethylhexyl) ester; benzophenone derivatives such as 2-hydroxy-4-methoxy benzophenone; derivatives of 3-benzylidene camphor such as 3-benzylidene camphor.

Preferred oil-soluble UV filters are benzophenone-3, butyl methoxybenzoylmethane, octyl methoxycinnamate, octyl salicylate, 4-methylbenzylidene camphor, homosalate and octyl dimethyl PABA.

Water-soluble UVB filters are e.g. sulphonic acid derivatives of benzophenone or of 3-benzylidene camphor, or salts such as the Na salt or K salt of 2-phenylbenzimidazol-5-sulphonic acid.

UVA filters include derivatives of dibenzoylmethane such as 1-phenyl-4-(4′-isopropylphenyl) propane-l, 3-dione.

The inventive cosmetic compositions can e.g. be used in the form of suncreams, sun gels, after-sun products, day creams, night creams, masks, body lotions, cleansing milk, body powder, eye cosmetics, hair masks, hair conditioners, hair shampoos, shower gels, shower oils, bath oils and in decorative cosmetic products such as deo sticks, perfume sticks, lipsticks, gels, eyeshadows, compact products such as compact powder or compact wax, rouge, foundation, make-up, etc. The aforesaid products are manufactured in a way known to those skilled in the art.

Creams, gels, masks, lotions, eye cosmetics, make-ups are particularly preferred since these forms of application are in most cases applied regularly. The effects achieved, i.e. a reduction or flattening of wrinkles, can e.g. be seen in the photos. Noticeable improvements can already be observed after 7 days of applying the respective product to the facial skin twice a day.

### BRIEF DESCRIPTION OF THE DRAWINGS

The invention will now be explained in more detail by means of examples. All quantities are in percent by weight unless indicated otherwise.

In the attached drawing,

FIG. 1: shows a photo of a skin area treated in vivo with the cream according to Example 2, (A) prior to treatment and (B) after 7 days of treatment.

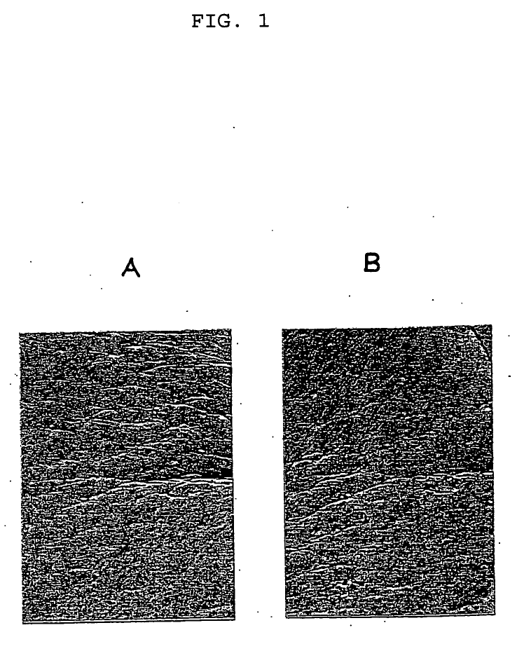

### DETAILED DESCRIPTION OF THE INVENTION

### Example 1

Skin Cream for Normal Skin

Phase A

| Water | up to 100% |
|---|---|
| Glycerine | 3.0 |
| Propylene Glycol | 3.0 |

Phase B

| | |
|---|---|
| Silicone Gel (Dimethicone & PEG 15 | 4.8 |
| Dimethicone Crosspolymer) | |
| Dimethicone Silicone | 11.0 |

Phase C

| | |
|---|---|
| Vitamin Mixture (B2, B6, B12, C, E, D) | 1.0 |
| Ethanol | 5.0 |
| RPF Complex* | 0.5 |
| Mentha Extract | 2.5 |
| Passiflora Extract | 0.5 |
| Papaver Extract | 1.0 |
| Myrtus Extract | 0.5 |
| T102 | 0.5 |
| Vitamin A Palmitate | 0.05 |
| Perfume | 0.5 |
| Preservative | 0.5 |

*Active complex according to Example 1 of WO 99/66881

Phases A and B were prepared separately and, in doing so, both were heated to approx. 80° C. The two phases were combined with one another while stirring and homogenized for 20 minutes. The mixture was cooled down to 60° C., homogenized for approx. 5 minutes, then cooled down to 50° C. and homogenized for another 5 minutes. Subsequently, the mixture was cooled down to 35° C. and Phase C, which had been prepared separately at temperatures below 35° C., was added and homogenized for some minutes again.

### EXAMPLE 1a

0.8% of the hexapeptide acetyl-Glu-Glu-Met-Gln-Arg-Arg-NH² was added to Phase C.

### EXAMPLE 2

Body Cream I

Phase A

| Water | up to 100% |
|---|---|
| Glycerine | 10.0 |
| Propylene Glycol | 3.0 |

Phase B

| | |
|---|---|
| Silicone Gel (Dimethicone & PEG 10 | 5.8 |
| Dimethicone Crosspolymer) | |
| Dimethicone Silicone | 12.0 |

Phase C

| | |
|---|---|
| Vitamin Mixture | 1.0 |
| Alcohol | 10.0 |
| RPF Complex* | 0.5 |
| Mentha Extract | 2.0 |
| Passiflora Extract | 0.3 |
| Papaver Extract | 2.0 |
| Myrtus Extract | 1.5 |
| Vitamin A Palmitate | 0.5 |
| Perfume | 0.5 |
| Preservative | 0.5 |

*Active complex according to Example 2 of WO 99/66881

Processing was done as in Example 1.

### EXAMPLE 2a

1.3% of the hexapeptide was added to Phase C.

### EXAMPLE 3

Cream for Dry Skin

Phase A

| Water | up to 100% |
|---|---|
| Glycerine | 5.0 |
| 10Propylene Glycol | 3.0 |

Phase B

| | |
|---|---|
| Silicone Gel (Dimethicone & PEG 10 | 6.9 |
| & PEG 15 Dimethicone Crosspolymer) | |
| Dimethicone Silicone | 14.8 |

Phase C

| | |
|---|---|
| Vitamin Mixture | 1.0 |
| Alcohol | 8.0 |
| RPF Complex* | 0.8 |
| Mentha Extract | 2.5 |
| Passiflora Extract | 0.5 |
| Papaver Extract | 1.2 |
| Myrtus Extract | 0.5 |
| Ti02/Si02 Pigment | 0.5 |
| Vitamin A Palmitate | 0.1 |
| Perfume | 0.5 |
| Preservative | 0.5 |

*Active complex according to Example 1 of WO 01/26617 Processing was done as in Example 1.

### EXAMPLE 3a

1.5% of the hexapeptide and 1.1% of the RPF complex according to Example 2 of WO 99/66881 were added to Phase C.

### EXAMPLE 4

Body cream II

Phase A

| Water | up to 100% |
|---|---|
| Glycerine | 10.0 |
| Propylene Glycol | 3.0 |

Phase B

| | |
|---|---|
| Silicone Gel (Dimethicone & PEG 10 | 5.8 |
| & PEG 15 Dimethicone Crosspolymer) | |
| Dimethicone Silicone | 12.0 |

Phase C

| | |
|---|---|
| Vitamin Mixture | 1.0 |
| Alcohol | 10.0 |
| Hexapeptide* | 1.0 |
| Mentha Extract | 2.0 |
| Passiflora Extract | 0.3 |
| Papaver Extract | 2.0 |
| Myrtus Extract | 1.5 |
| Vitamin A Palmitate | 0.5 |
| Perfume | 0.5 |
| Preservative | 0.5 |

*Acetyl-Glu—Glu-Met-Gln-Arg—Arg-NH2 Processing was done as in Example 1.

### EXAMPLE 4a

No hexapeptide was added to Phase C.

### EXAMPLE 5

Comparative Test Regarding Moisture Improvement

The moisture in the skin of 18 female test persons with dry mixed skin was measured using a comeometer. The measurements were taken with a Comeometer CM 825 (Courage & Khazaka, Germany) at 22° C. and 56% relative air humidity. The different creams were applied 2 hours after the skin had been cleansed. The following table shows the results as regards moisturizing in % as average values.

TABLE 1

| | | Cream of | | Cream of | | Cream of Expl. 1 | |
|---|---|---|---|---|---|---|---|
| | | Example 1 | | Example 2a | | without extracts | |
| Time | | Average | Increase | Average | Increase | Average | Increase |
| before | | 42.5 | — | 40 | — | 42.5 | — |
| 0.5 | h | 58 | +36% | 51 | +28% | 52 | +22% |
| 2 | h | 58 | +36% | 53 | +33% | 53 | +24% |
| 6 | h | 56 | +32% | 54 | +35% | 52 | +22% |
| 24 | h | 60 | +41% | — | — | 45 | +5% |
| 2 | weeks | 62 | +45% | 59 | +48% | — | — |
| 4 | weeks | 65 | +53% | 63 | +54% | — | — |

The comparison shows that the cream according to Example 1 of the present invention containing the aforedescribed complex has a moisturizing effect which is noticeably better than that of a cream without this complex. The comparative cream without the complex still showed an increased moisture value after 8 hours, but fell to nearly zero after 24 h, while the inventive cream had increased moisture values of +41% It is particularly significant that these values are maintained and even increased over a period of time of 4 weeks.

### EXAMPLE 6

Comparative Tests Regarding the Anti-Wrinkle Effect

Tests were carried out in order to prove the anti-wrinkle effect. The test included 21 male and female test persons aged between 42 and 61 years. A microrelief of parts of the facial skin (eye area, comers of the mouth, nose area) was taken using a silicone mass, said mass was left to harden and the negative relief obtained was measured electrooptically as regards the depth and number of wrinkles. Immediately after the microrelief had been taken, a cream was applied to the face of the test persons for the first time, which cream was then applied repeatedly twice a day in an amount of approx. 2 g/cm².
Group 1: 12 test persons were treated with the cream of Example 1;
Group 2: 6 test persons were treated with the cream of Example 2a;
Group 3: 3 test persons were treated with a cream which consisted only of the basic formulation of Example 1 without active agents (placebo).

Control measurements were carried out on the 14th and 28th day after the first measurement by taking the microrelief of the same skin areas of the individual test persons. During this time, no test person was subjected to particularly intense solar radiation. The values determined are statistical average values for a selected area of the microrelief.

TABLE 2

| | Number of test persons | | |
|---|---|---|---|
| | Group 1 | Group 2 | Group 3 |
| Reduction of wrinkle depth | | | |
| after 14 days | | | |
| by 10-30% | 8 | 4 | 1 |
| by 30-50% | 4 | — | — |
| Reduction of wrinkle depth | | | |
| after 28 days | | | |
| by 10-30% | 2 | 1 | 1 |
| by 40-50% | 7 | 3 | — |
| >50% | 2 | 2 | — |

Group 1 shows a very good reduction of the wrinkle depth after 28 days for 75% of the test persons. Compared to Group 1, the inventive composition according to Example 2a, tested in Group 2, shows a significant additional improvement, which can be put down to the interaction with the hexapeptide.

Now that the invention has been described,

We claim:

1. An anti-wrinkle cosmetic comprising the following ingredients in a W/O silicone oil system
0.05 to 3.0% by weight of an extract from Papaver,
0.05 to 2.0% by weight of an extract from Passiflora,
0.05 to 3.0% by weight of an extract from Mentha,
0.05 to 3.0% by weight of an extract from Myrtus,
in addition to usual cosmetic auxiliaries, carriers, active agents or mixtures thereof, which make up the remainder up to 100%.

2. The anti-wrinkle cosmetic according to claim 1, wherein said cosmetic contains 0.5 to 15% by weight of a solution of the hexapeptide acetyl-Glu-Glu-Met-Gln-Arg-Arg-NH 2 as an additional active agent with the concentration of the pure peptide in said solution being 0.5 g/l.

3. The anti-wrinkle cosmetic according to claim 1, wherein said extract from Papaver is an extract from the seeds of P. adults, P. rhoeas, P. incarnata, P. laurifolia, P. quadrangularis, P. somniferum or mixtures thereof.

4. The anti-wrinkle cosmetic according to claim 1, wherein said extract from Mentha is an extract from M. aquatica, M. arvensis, M. piperita, M. pulegium, M. rotundifolia, M. viridis or mixtures thereof.

5. The anti-wrinkle cosmetic according to claim 1, wherein the silicone oils of said silicone oil system are selected from the group consisting of cyclohexasiloxane, cyclopentasiloxane, cyclotetrasiloxane, dimethicone and mixtures thereof.

6. The anti-wrinkle cosmetic according to claim 1, wherein said cosmetic contains Dimethicone & PEG 10 Dimethicone Crosspolymer & PEG 15 Dimethicone Crosspolymer as silicone gel, together with one or several silicone oil (s).

7. The anti-wrinkle cosmetic according to claim 1, wherein said cosmetic contains 0.2 to 1.5% by weight of an active complex, relative to the cosmetic's total weight, as an additional active agent, which complex consists of 0.1 -10% by weight of an extract from the bark of Quebracho blanco, 0.1-10% by weight of a silkworm extract, 0.1-5% by weight of a hydrogel, 0.1-30% by weight phospholipids and 45-99.6% by weight water relative to the total weight of the active complex.

8. The An anti-wrinkle cosmetic according to claim 1, wherein said cosmetic contains neither pigments nor powder.

9. The anti-wrinkle cosmetic according to claim 1, wherein said cosmetic comprises 0.8-2.4% by weight of an extract from Papaver, 0. 1-0.9% by weight of an extract from Passiflora, 1.2-2.6% by weight of an extract from Mentha, 0.4-2.8% by weight of an extract from Myrtus and 0.5-2.0% by weight of a solution of the hexapeptide acetyl-Glu-Glu-Met-Gln-Arg-Arg-NH 2 in a cosmetic composition.
